(19) United States
(12) Patent Application Publication
(10) Pub. No.: US 2026/0065471 A1
(43) Pub. Date: Mar. 5, 2026

(54) METHOD FOR BRAIN/HEAD IMAGE CREATION

(71) Applicant: Eped, Inc., Kaohsiung City (TW)

(72) Inventors: Ta-Ko Huang, Kaohsiung City (TW); Jerry T. Huang, Kaohsiung City (TW)

(21) Appl. No.: 19/318,839

(22) Filed: Sep. 4, 2025

(30) Foreign Application Priority Data
Sep. 4, 2024 (TW) 113133502

(51) Int. Cl. G06T 7/00; G16H 30/40

(52) U.S. Cl. CPC G06T 7/0012; G16H 30/40; G06T 2207/10081; G06T 2207/30016

(57) ABSTRACT

The present invention provides a method for brain image creation and a method for head image creation, which can be used to assist in the positioning of non-invasive brain treatment device (such as magnetic stimulation (TMS) or electrical stimulation, etc.) in brain areas. The method for brain image creation of the present invention performs proportional correction according to a 3D head image inputted (such as computer tomography CT or magnetic resonance imaging MRI or ultrasound), so that the relative position between the skull and the brain is clearer. Wherein, the method for brain image creation is to perform proportional correction on the 3D head image inputted to obtain a brain image through a computing device; the proportional correction step includes an initialization step, a reference model construction step, a region growth step and a model post-processing step.

### BACKGROUND

### Related Application

This non-provisional application claims priority under 35 U.S.C. § 119(a) to patent application No. 113133502 filed in Taiwan, R.O.C. on Sep. 4, 2024, the entire contents of which are hereby incorporated by reference.

### Technical Field

The present invention is a method for analyzing and organizing images to obtain brain images with improved resolution.

### RELATED ART

Non-invasive brain treatment devices such as Transcranial Magnetic Stimulation (TMS) or repetitive Transcranial Magnetic Stimulation (rTMS) are both non-invasive neuromodulation technologies that use magnetic fields to regulate brain activity. Since rTMS refers to stimulation in a repetitive manner, it has the same setting as TMS. The main uses of TMS and rTMS include the treatment of depression, anxiety (including Obsessive-Compulsive Disorder, OCD), Post-Traumatic Stress Disorder (PTSD), Parkinson's disease, stroke rehabilitation, chronic pain (for example: neuropathic pain and fibromyalgia), insomnia, epilepsy and many other diseases related to brain nerves.

The main principle of non-invasive brain treatment devices such as TMS or rTMS is to repeated magnetic stimulation is applied to specific areas of a brain to change the activity mode of neurons, thereby achieving a therapeutic effect. During the treatment, the patient usually sits in a chair with his head fixed and magnetic coils placed at specific locations on the scalp. The magnetic field penetrates the scalp and triggers neuronal activity. When choosing a TMS or rTMS placement, healthcare professionals make decisions based on the goals of treatment and the specific circumstances of the patient. For example: Depression: Usually stimulates the left dorsolateral prefrontal cortex (DLPFC), which is related to emotion regulation. Obsessive-compulsive disorder: Stimulation of the brain's medial prefrontal cortex or the cortical-basal ganglia-thalamic circuit connected to it. Stroke rehabilitation: Based on the symptoms after a stroke, the contralateral cerebral cortex of the damaged area is stimulated to promote functional recovery.

However, how to select a suitable location for positioning is a problem that needs to be overcome. The systems currently in use include:
(1) “10-20 system”: This is an internationally standardized electroencephalogram (EEG) positioning system used to locate specific points on the scalp. It is often used clinically to help determine the stimulation locations of non-invasive brain treatment devices such as TMS or rTMS.
(2) Anatomically guided positioning: Using images of the patient's brain (such as MRI) to precisely locate the area that needs stimulation, which can improve the accuracy and effectiveness of treatment.
(3) Trial and error method: When starting treatment, the doctor will monitor the patient's response and adjust the position of the magnetic coil and stimulation parameters based on the treatment effect and side effects.

However, all of the above methods require brain positioning. Regardless of which method is used, patients will experience discomfort when searching for the location. Although doctors can refer to various images to infer the location, they are still unable to quickly find the corresponding brain area from the outside of the skull. Therefore, how to assist non-invasive brain treatment devices such as TMS or rTMS in finding the corresponding setting location outside the skull is a very important issue.

### SUMMARY

The present invention provides a method for brain image creation and a method for head image creation, which are useful for positioning a non-invasive brain treatment device. Among them, the method for brain image creation is to perform proportional correction according to a 3D head image inputted (such as computer tomography CT or magnetic resonance imaging MRI or ultrasound) to make the relative position between the skull and the brain clearer. The method for brain image creation is to perform proportional correction on a head image inputted to obtain a brain image through a computing device; the proportional correction step includes an initialization step, a reference model construction step, a region growth step and a model post-processing step.

The initialization step is to process the head image inputted, filter out a part of a grayscale image in the head image, and define the head image processed as an initialization image. The reference model construction step is to perform brightness analysis on the initialization image at each coordinate position of image pixels, and then calculate an image centroid based on thereof, and establish a geometric reference object based on the image centroid. The region growth step calculates the boundary position according to the grayscale status of the surface image of the geometric reference object, and defines the image after this step as an image with a boundary position. The model post-processing step is to smooth the image with the boundary position.

After completing the above steps, the brain image is created. The advantages of creating brain image in this way include:
1. Quickly complete the creation of the brain model. This method can greatly shorten the time of brain model creation and thus improve efficiency.
2. It is helpful for the positioning of non-invasive brain treatment device: High-precision brain imaging model is helpful for the positioning of non-invasive brain treatment device and improve the safety of surgery.
3. No need to inject developer: no additional risk to patients.
4. Applications of multi-purposes: In addition to TMS or rTMS or electrical stimulation, these images can also be used for research, teaching and other medical applications.
5. Reduce costs: Through fast and efficient image creation, the consumption and cost of medical resources can be reduced.

These advantages make this method one of the preferred methods for creating brain images in modern medicine, helping to improve the overall quality of medical care.

Furthermore, the image processing described in the initialization step of the present invention is to form an image histogram, and obtain a maximum brightness value and a minimum brightness value according to the image histogram. A low critical brightness value and a high critical brightness value are set between the maximum brightness value and the minimum brightness value, and each pixel between the low critical brightness value and the high critical brightness value in the head image is taken out to form the initialization image. In this way, the pixels that need to be processed can be reduced, thereby improving the efficiency of calculation.

Furthermore, in the reference model construction step, the step of performing brightness analysis on the initialization image at each coordinate position of the image pixels is: recording the coordinate position and a brightness value of each pixel, wherein the x coordinate of the image centroid is the sum of the x coordinates of all pixels multiplied by the brightness value of the coordinate, and then divided by the sum of brightness values of all pixels, and the y coordinate of the image centroid is the sum of the y coordinates of all pixels multiplied by the brightness value of the coordinate, and then divided by the sum of brightness values of all pixels. By obtaining the position of the image centroid, a reference model is created and used as the basis for regional growth.

Furthermore, in the reference model construction step, the volume of the geometric reference object can be calculated according to the number of pixels, and a median brightness can be obtained according to the pixels of the geometric reference object, and the radius of a sphere can be calculated based on the median brightness. This makes the creation of geometric reference objects closer to the actual situation.

Based on the above-mentioned method for brain image creation, the present invention further provides a method for head image creation, wherein at least one registration point is set between the head model and the brain image, and then the head model is deformed and scaled according to the proportion of the brain image, and the brain image is combined with the head model according to the registration point, so that the relative position of the head model and the brain image is better defined.

In order to enable the examiner to have a further understanding of the purpose, technical features and effects of the present invention, the following embodiments are provided with accompanying drawings and detailed descriptions.

### BRIEF DESCRIPTION OF THE DRAWINGS

FIG. 1 is a flow chart of an embodiment of the present invention.

FIG. 2 is a flow chart of another embodiment of the present invention.

### DETAILED DESCRIPTION

This embodiment is a method for a brain image creation and a method for a head image creation, in particular, a method for processing CT (computed tomography), MRI (magnetic resonance imaging) or X-ray images to produce a brain image, which can be used for positioning of non-invasive brain treatment device or for brain treatment teaching applications. Furthermore, the method for brain image creation uses a head image inputted to perform proportional correction, making the relative position between the skull and the brain clearer. When performing non-invasive brain treatment device positioning such as rTMS treatment, it will be able to quickly and accurately align to reduce the discomfort when searching for the position.

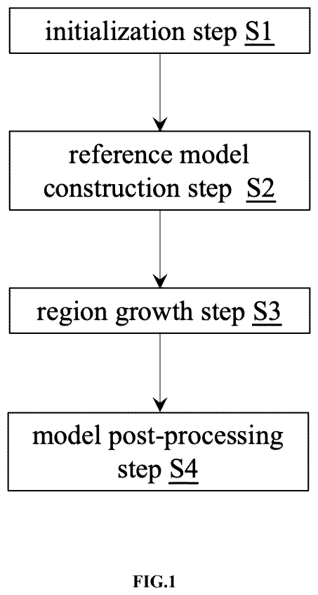

The method for brain image creation is to obtain a brain image by performing a proportional correction step on the head image inputted through a computing device; wherein the proportional correction step includes an initialization step S1, a reference model construction step S2, a region growth step S3 and a model post-processing step S4, as shown in FIG. 1.

The initialization step S1 is to perform image processing on the head image inputted. Filter and select a part of a grayscale image in the head image, and define the head image processed as an initialization image. Wherein, the image processing is to obtain an image histogram of the head image, and obtain a maximum brightness value and a minimum brightness value according to the image histogram. The image histogram is a graph used to represent the distribution of the number of pixels of each gray level (or color) in an image, and is used to understand the brightness distribution of the image. Converting an image into an image histogram is a known technology, so the technical means for converting into an image histogram will not be described in detail in this embodiment, this is described here.

After obtaining the image histogram converted, a high critical brightness value and a low critical brightness value are set between the maximum brightness value and the minimum brightness value on the image histogram, and each pixel (an image) in the head image located between the low critical brightness value and the high critical brightness value is taken out (or deleting each pixel that is not between the low critical brightness value and the high critical brightness value), so as to form the initialization image. In this way, the number of pixels to be processed can be reduced and the efficiency of calculation can be improved.

The reference model construction step S2 is to perform brightness analysis on the initialization image at each coordinate position of image pixels, and then calculate an image centroid based on thereof, and establish a geometric reference object based on the image centroid. The calculation method of the image centroid is disclosed in this embodiment as one method, but is not limited thereto. This embodiment records the coordinate position and a brightness value of each pixel. Then, the x coordinate of the image centroid is the sum of the x coordinates of all pixels multiplied by the brightness value of the coordinate, and then divided by the sum of the brightness values of all pixels. The y coordinate of the image centroid is the sum of the y coordinates of all pixels multiplied by the brightness value of the coordinate, and then divided by the sum of brightness values of all pixels. By obtaining the position of the image centroid, the geometric reference object is established and used as the basis for region growth. Furthermore, the volume of the geometric reference object can be calculated based on the number of pixels, and a median brightness can be obtained based on the pixels of the geometric reference object, and the radius of a sphere can be calculated based on the median brightness, so that the establishment of the geometric reference object is closer to the actual situation. Furthermore, the geometric reference object may be a polygon with more than 20 faces, or a polygon derived from the sphere.

The region growth step S3 calculates the boundary position based on the grayscale status of the surface image of the geometric reference object, and defines the image after this step as an image with a boundary position. This step is based on the geometric reference object, and searches for adjacent grayscale values within the image range (eg, each surface or each segmented range), and then forms a connection line. Furthermore, the adjacent grayscale can be found within the image range, and a normal line can be established to calculate the approximate grayscale value outside the image range.

The model post-processing step S4 is to smooth the image with the boundary position, so as to be closer to the actual image of the brain.

After completing the above steps, the brain image is created. By creating a brain image in this way, it is convenient to locate the non-invasive brain treatment device, that is, it is easy to choose the location on the scalp where the brain treatment device is placed. When selecting the placement of a non-invasive brain treatment device, medical professionals can place it based on the brain images completed by the present invention, which will overcome the discomfort caused by traditional one-by-one searches.

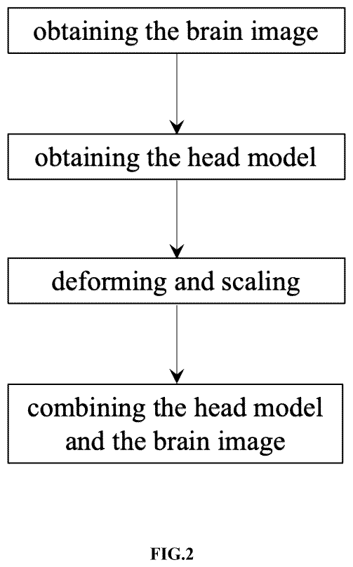

Based on the above method for brain image creation, an embodiment of the method for head image creation is further provided, as shown in FIG. 2, wherein at least one registration point is first set between the head model and the brain image, and then the head model is deformed and scaled according to the proportion of the brain image, and then the brain image is combined with the head model according to the registration point, so that the relative position of the head model and the brain image is more clearly defined, which provides a safer auxiliary for the non-invasive brain treatment device TMS or rTMS of brain treatment. At the same time, when used in medical teaching, it can help students better understand how to quickly find the appropriate position for using.

However, the above description is only a preferred embodiment of the present invention and is not intended to limit the scope of patent protection of the present invention. Therefore, all equivalent changes made by using the contents of the present invention's description and drawings are also included in the scope of protection of the present invention and are hereby stated.

### REFERENCE SIGN LIST

[symbol] method for brain image creation
[symbol] initialization step
[symbol] reference model construction step
[symbol] region growth step
[symbol] model post-processing step
[symbol] image centroid
[symbol] image histogram
[symbol] deform and scale

What is claimed is:

1. A method for brain image creation, performing a proportional correction step on a head image inputted to obtain a brain image through a computing device; the proportional correction step comprising:
an initialization step, performing image processing on the head image inputted, first filtering out a part of a grayscale image in the head image and defining the head image processed as an initialization image;
a reference model construction step, performing brightness analysis on the initialization image at each coordinate position of image pixels, and then calculating an image centroid based on thereof, and establishing a geometric reference object based on the image centroid;
a region growth step, calculating a boundary position based on a grayscale status of a surface image of the geometric reference object, and defining the image after this step as an image with the boundary position; and
a model post-processing step, smoothing the image with the boundary position.

2. The method for brain image creation according to claim 1, wherein the image processing described in the initialization step is to form an image histogram, and obtain a maximum brightness value and a minimum brightness value according to the image histogram; further, a low critical brightness value and a high critical brightness value are set between the maximum brightness value and the minimum brightness value, and an image in the head image located between the low critical brightness value and the high critical brightness value is taken out to form the initialization image.

3. The method for brain image creation according to claim 1, wherein in the reference model construction step, the step of performing brightness analysis on the initialization image at each coordinate position of the image pixels is: recording the coordinate position and a brightness value of each pixel, wherein the x coordinate of the image centroid is the sum of the x coordinates of all pixels multiplied by the brightness value of the coordinate, and then divided by the sum of the brightness values of all pixels, and the y coordinate of the image centroid is the sum of the y coordinates of all pixels multiplied by the brightness value of the coordinate, and then divided by the sum of the brightness values of all pixels.

4. The method for brain image creation according to claim 3, wherein in the reference model construction step, the volume of the geometric reference object is further calculated based on the number of pixels, and a median brightness is obtained based on the pixels of the geometric reference object, and the radius of a sphere is calculated based on the median brightness.

5. A method for head image creation, comprising:
obtaining a brain image by the method according to claim 1;
obtaining a head model and establishing at least one registration point of the head model and the brain image; and
deforming and scaling the head model according to the proportion of the brain image and combining the brain image with the head model according to the registration point.
